(19) United States
(12) Patent Application Publication
(10) Pub. No.: US 2017/0290717 A1
(43) Pub. Date: Oct. 12, 2017

(54) Flexible film for film strips

(71) Applicant: MONDI GRONAU GMBH, Gronau (DE)

(72) Inventors: Marcus Schoenbeck, VERSMOLD (DE); HENNER SOLLMANN, GRONAU (DE)

(21) Appl. No.: 15/480,470

(22) Filed: Apr. 6, 2017

(30) Foreign Application Priority Data
Apr. 8, 2016 (EP) 16164547.8

(51) Int. Cl. A61F 13/49; A61L 15/42; A61L 15/24

(52) U.S. Cl. CPC A61F 13/49015; A61L 15/24; A61L 15/42

(57) ABSTRACT

An elastic film having an elastic film layer based on styrene block copolymer, such that the elastic film layer is formed from a first styrene block copolymer and a second styrene block copolymer without the addition of polystyrene. The first styrene block copolymer has a styrene portion of less than 25% by weight and the second styrene block copolymer has a styrene portion of more than 26% by weight. A ratio of the first styrene block copolymer to the second styrene block copolymer is between 6:1 and 2:3.

### CROSS-REFERENCE TO A RELATED APPLICATION

The invention described and claimed hereinbelow is also described in European Patent Application EP 16 164 547.8, filed on Apr. 8, 2016. The European Patent Application, the subject matter of which is incorporated herein by reference, provides the basis for a claim of priority of invention under 35 U.S.C. 119(a)-(d).

### BACKGROUND OF THE INVENTION

The invention relates to an elastic film having an elastic film layer on the basis of styrene block copolymer. Films of this type are utilized for elastic laminates in the field of personal hygiene, for example.

Conventionally, elastic films having an elastic film layer can form, for example, the elastic component of a diaper closure of a disposable diaper, wherein specific requirements are placed specifically on the elastic properties of the elastic film.

Diapers, for example, for babies and young children, are produced as disposable mass-produced articles, and optimizing such mass production relies upon an efficient utilization of material and upon low production costs.

Simultaneously, however, a high degree of functionality also is necessary, so that a high degree of security as well as reliable protection of the diaper-wearing person can be ensured under all usage conditions. Overnight, large quantities of excrement must be absorbed and securely retained by a diaper when worn, wherein adverse effects on the skin of a wearer should be avoided to the greatest extent possible. Disposable diapers are therefore provided with elastic elements and sections for sealing at the waist and the legs of a user. At the same time, however, a high level of wearing comfort must be ensured during movement by the user. Constrictions and skin irritations caused by the elastic sections and elements should be avoided. Elastic laminates, which comprise layers of nonwoven fabric, and an elastic core therebetween, on their opposite outer sides, are known for forming elastic lateral parts and lateral diaper closures.

According to the prior art, the elastic core can be formed, for example, from an elastic film incorporated over the entire surface, or from elastic strands. An elastic laminate for diaper closures comprising nonwoven-fabric layers forming two opposite outer sides and comprising strips of an elastic film, which extend in a stretching direction and are incorporated between the nonwoven-fabric layers by adhesive, is known from EP 1 928 380 B1. An elastic layer without cover layers can be incorporated either over the entire surface or in the form of strips between the nonwoven-fabric layers, wherein the material formed in this way can be initially stretched only by application of a substantial amount of force, due to the nonwoven-fabric layers, and is therefore initially subjected to an activation.

In the case of activation carried out using ring rollers, which are known, for example, from U.S. Pat. No. 4,834,741, the structure of the outer nonwoven-fabric layers is stretched and is usually also partially destroyed. The activated elastic laminate is subsequently easy to stretch during use, at least up to the stretching limit predetermined by the activation. One further method step is necessary for the activation in this case, wherein, depending on the material of the nonwoven-fabric layers, structural damage cannot be ruled out during the activation, which can result in an inadvertent over-stretching or even to tearing during a use of the elastic laminate, because, depending on the specific embodiment, the over-stretched nonwoven-fabric layers can only make a small contribution to the stability of the elastic laminate. The necessary stability of the elastic laminate is provided instead by the strips incorporated between the nonwoven fabric, and therefore the strips must have sufficient tensile strength.

It also is known from the prior art to incorporate all-over films or individual elastic filaments in a stretched state between two layers of nonwoven fabric, wherein the nonwoven-fabric layers contract into pleats when the tensile forces are discontinued, and therefore a particularly soft and pleasant fit results when such a material is used for a diaper. In addition, the advantage results that the degree of the preload of the incorporated elastic elements predefines a distinct stretching limit. The elastic laminate can be stretched by the same extent that it had previously contracted after the tensile forces are discontinued. The structure of the nonwoven-fabric layers is not substantially changed in this case. The nonwoven-fabric layers have high stability, even when the stretching limit predefined by the preload has been reached, and are not preliminarily damaged in terms of their structure by an activation.

The incorporation of individual filaments in a stretched state is known, for example, from EP 1 179 330 B1, EP 1 610 950 B1, EP 1 707 351 A1 and EP 1 425 171 B1. The handling of the individual filaments during the production, however, is complex.

### SUMMARY OF THE INVENTION

The present invention overcomes the shortcomings of known arts, such as those mentioned above.

The present invention proves an elastic film with an elastic film that is easy to stretch, in the production direction and at least after an initial activation, and which has a high degree of elasticity.

Proceeding from an elastic film having the features described at the outset, it is therefore provided according to the invention that the elastic film layer is formed from a first styrene block copolymer and a second styrene block copolymer without the addition of polystyrene. The first styrene block copolymer has a styrene portion of less than 25% by weight and the second styrene block copolymer has a styrene portion of more than 26% by weight. The ratio of the first styrene block copolymer to the second styrene block copolymer is between 6:1 and 2:3.

Due to the combination of two styrene block copolymers, a particularly easy stretchability combined with sufficient strength can be implemented.

It is preferably provided that the first styrene block copolymer has a styrene portion of less than 22% by weight, in particular between 10% to 20% by weight, and a hardness of less than 45 Shore A, where the second styrene block copolymer has a styrene portion of more than 26% by weight, in particular between 30% and 40% by weight, and a hardness of more than 45 Shore A. Proceeding therefrom, the ratio of the first, soft styrene block copolymer to the second styrene block copolymer preferably lies in a range between 5:1 and 1:1.

Without the addition of polystyrene and due to the first portion of the first styrene block copolymer of at least 40% as compared to the second styrene block copolymer, the elastic film layer has an easy stretchability but also a low tensile strength and high tackiness.

Due to the composition of the elastic film layer according to the invention, good elastic properties are specifically achieved even in the machine direction (production direction). The film, therefore, also can be processed and situated in such a way that the film is stretched along the machine direction, during proper use, and is elastic, whereas the stretching of the elastic film according to the prior art is often provided in the transverse direction and, therefore, the stretching properties in the transverse direction are significant and advantageous.

Strips can be cut from the elastic film according to the invention, for example, along the machine direction, and therefore the orientation of the strips can also correspond to the eventual direction of the stretching if the cut strips are processed, as continuous material, to form a laminate. As described in greater detail in the following, strips of the elastic film can be introduced between two layers of nonwoven fabric, for example.

Under consideration of these method-related and structural specifications, the stretchability of the elastic film in the machine direction i.e., along the polymer strands oriented during the extrusion, is advantageous. In this context, it is assumed that due specifically to the combination of two styrene block copolymers, which differ in terms of their mechanical properties, and the omission of polystyrene as a usual additive, the necessary elasticity can be achieved specifically in the machine direction.

According to an embodiment, the invention provides that the elastic film layer is situated between two non-elastic cover layers in the case of a three-layered configuration formed by co-extrusion. This three-layered configuration is advantageous for several reasons. Initially, the elastic film formed in this way can be readily rolled up, transported, and unrolled without becoming blocked in the rolled-up state due to the tackiness of the elastic film layer might otherwise result from the use based on styrene block copolymers. In addition, the cover layers can stabilize the elastic film layer both during the co-extrusion process and during the subsequent processing. During a subsequent processing, the elastic film can be rolled up and unrolled, for example, without excessive stretching, provided the structure of the cover layers is not damage.

During the co-extrusion, the non-elastic cover layers also can effectuate a delimitation and stabilization of the soft styrene block copolymer. Specifically, when the elastic film comprises non-elastic cover layers, a preliminary stretching before connection to the nonwoven-fabric layers is advantageous in the above-described method to activate the elastic film by over-stretching the cover layers. The advantage then also results that the cover layers, after such an activation, cannot adversely affect the elastic properties of the elastic film to a substantial extent.

The invention also provides that each of the non-elastic cover layers has only a small thickness, for example, between 1.5 μm and 6 μm, and in particular, between 2 μm and 4 μm, e.g., 3 μm. Initially, it can be ensured by way of a small thickness that a preliminary stretching for activating the elastic film is easily possible and the elastic properties of the elastic film are not adversely affected by the cover layers to a substantial extent. In addition, the cover layers are provided to stabilize the elastic film layer based on styrene block copolymers during the co-extrusion and the processing, and therefore a small thickness also is sufficient for this purpose. Finally, the layers are preferably designed to be only as thick as necessary for the particular function, to save material.

The non-elastic cover layers can be formed, for example, from polyolefin, in particular, from polyethylene, polypropylene, or mixtures of polyethylene and polypropylene. Polyolefins are inexpensive and are also usually relatively easy to stretch.

The thickness of the elastic film layer is preferably between 30 μm and 60 μm, and more preferably between 40 μm and 50 μm, e.g., 45 μm.

An intent of the invention is to achieve a particularly uniform and easy stretchability of the film and, if applicable, also of an elastic laminate formed from the elastic film. If strips of the elastic film are incorporated under preload between two layers of nonwoven material, a stretching limit of the elastic laminate is determined by this preload. As a result, the elastic film and, in particular, the elastic film layer should also have good elastic return properties but, overall, only need to have a low tensile strength. When the stretching limit predefined by the preload is reached, a further stretching is limited by the outer nonwoven-fabric layers, which can usually be perceived immediately by the user in the form of a rapid increase in force upon further stretching.

In addition to an easy stretchability, the elastic laminate should also have sufficient holding forces. This can be ensured by way of the elastic film and an elastic laminate formed therefrom having largely elastic properties and by way of the energy applied during the stretching being largely recoverable during a contraction.

For this purpose, EP 1 928 380 B1 defines an energy recovery value; the full scope of the definition thereof is referred to within the scope of the present invention. A feature of the invention may derive from the test method set forth therein. According thereto, an MTS alliance RT/1 test system can be utilized for determining tensile forces to determine a hysteresis in a stretch test, based on which the energy recovery value is determined. The energy recovery value indicates the percentage of energy that is recovered during a stretching and a release of a test specimen by 200%. For this purpose, a test specimen is placed in a suitable test set-up, wherein the acting forces are recorded during the stretching of the subsequent contraction at a predefined test speed according to EP 1 928 380 B1. A hysteresis results in a usual displacement-force diagram in this case, wherein the energy recovery value results as a quotient of the integrals of the curves for the release, on the one hand, and for the stretching, on the other hand.

A theoretical energy recovery value of 1 therefore results for an ideal elastic material without hysteresis. Within the scope of the invention, the energy recovery value for the elastic film, in the machine direction, is preferably above 0.6, and most preferably above 0.68, for example. 0.75. As described above, a preliminary stretching can be provided for an initial activation of the elastic film.

In invention, after an initial activation by stretching by 500% and a return of the elastic film, the force required for a stretching by 100% is less than 1 N per 19 mm specimen width. In particular, the force lies between 0.5 N and 1 N per 19 mm specimen width, wherein the elastic film has the aforementioned properties preferably in the machine direction.

Correspondingly, after an initial activation by stretching by 500% and a return of the elastic film, the force required for one further stretching by 200% is less than 1.5 N per 19 mm and in particular, between 1 N and 1.5 N per 19 mm with respect to the machine direction.

Within the scope of the invention, a styrene-isoprene-styrene block copolymer (SIS) is provided as the first and the second styrene block copolymers.

In the case of a laminate formed from the elastic film according to the invention, it is provided that the strips are incorporated under preload between the nonwoven-fabric layers, and the nonwoven-fabric layers are pleated along the stretching direction in a relaxed state due to the return forces of the strips, which were incorporated under preload, i.e., said nonwoven-fabric layers are gathered. The thickness of the strips in the relaxed state is between 30 μm and 80 μm, and the width of the strips determined perpendicularly to the stretching direction is between 3 mm and 12 mm, and wherein the strips of the elastic film in the relaxed state cover between 10% and 80% of the total area, i.e., the total area of the laminate.

Typically, the strips extend equidistantly and with straight edges in parallel to one another. Preferably, the strips are adhered to the nonwoven-fabric layers directly, i.e., without any other intermediate layers.

Due to the incorporation of the strips of the elastic film in a stretched state, a clearly defined stretching limit is specified. After the tensile forces are discontinued after the laminate has been produced, the laminate contracts until the strips of the elastic film have returned to their starting length in the force-free state. According to the corresponding preloading of the strips of the elastic film, the elastic laminate can be stretched, for example, up to a stretching limit of stretching by between 30% and 300%, in particular, between 50% and 250%. Within the scope of the invention, the advantage results that the stability and tensile strength of the elastic laminate existing when the stretching limit is reached are substantially determined by the nonwoven-fabric layers. Even if the strips themselves are particularly easily stretched and, therefore, the elastic laminate can be stretched up to the stretching limit using a small amount of force, the nonwoven-fabric layers, the structure of which is not damaged by a usual activation according to the prior art, can provide a high level of strength. Therefore, in contrast to an activated elastic laminate, particularly easily stretched elastic films having a relatively low tensile strength can be utilized.

The elastic film in the relaxed state has a thickness of only 30 μm to 80 μm, wherein the width of the strips formed therefrom is between 3 mm and 12 mm. The strips of the elastic film therefore lie flat between the two layers of nonwoven fabric and therefore do not make a substantial contribution to the thickness. Due to the flat arrangement of the strips, the elastic properties of the material can also be optimally utilized, wherein, due to the surface coverage of between 10% and 80%, uniform elastic properties as well as an advantageous pleated structure of the nonwoven-fabric layers between the elastic strips in the relaxed state are achieved.

The arrangement of the nonwoven-fabric layers in the relaxed state also can be referred to as a ruffling effect, wherein a particularly soft and pleasant surface results, which also is optimally gentle on a user in the case of direct skin contact.

According to a refinement, the nonwoven-fabric layers are adhered all-over to the strips and to one another between the strips. Due to an all-over application of adhesive, a good adhesion of the elastic laminate is achieved, wherein a simple process also results. The all-over application of adhesive can take place, for example, by spray nozzles or a roller, wherein the complex creation of a pattern can be dispensed with.

According to another refinement, a hot melt adhesive is provided as the adhesive. This hot melt adhesive advantageously has elastic properties, so that the return of the strips, which have been incorporated under preload between the nonwoven-fabric layers, is not obstructed. Preferably, a hot melt adhesive based of styrene block copolymer, for example, styrene-isoprene-styrene block copolymer (SIS) or styrene-butadiene-styrene copolymer (SBS), is utilized. In addition, hot melt adhesives based on polyolefin elastometers also are options.

The strips of the elastic film, in the relaxed state, preferably cover between 30% and 65% of the total area. The distance between adjacent strips determined perpendicularly to the stretching direction in the relaxed state, measured from strip edge to strip edge, is advantageously between 3 mm and 12 mm, for example, 7 mm. A coverage of 50%, for example, therefore results when the width of the strips also corresponds to the distance between adjacent strips.

A method for producing an elastic laminate, in particular the above-described laminate, includes the steps of

extruding an elastic film having a thickness between 30 μm and 80 μm.

cutting the elastic film to form strips,

introducing the elastic strips in a stretched state in which said strips have been stretched by between 30% and 300%, in particular between 50% and 250%, and in which said strips are spaced apart from one another between two nonwoven-fabric layers,

laminating the nonwoven-fabric layers with the strips in the stretched state by use of adhesive, and

relaxing the laminate formed from the nonwoven-fabric layers and the strips.

The production direction in which the elastic film is extruded as monofilm or is preferably coextruded as a multilayered film, also is referred to as the machine direction. Preferably, the film is cut into strips in such a way that the individual strips also extend along the machine direction, which is advantageous with respect to the process at least because the strips are also formed as continuous material in this case and can be easily processed further.

A substantially greater force is often necessary to achieve a stretching by a film value specifically upon an initial stretching of the intermediate film than is the case in subsequent stretches. This applies, in particular, when, as is described in detail in the following, the elastic film is provided with thin, non-elastic cover layers, and therefore the elastic film itself must be initially activated to a certain extent to provide an easy stretchability. Against this background, it is provided that either the extruded film or the strips cut therefrom are preliminarily stretched before being introduced between the nonwoven-fabric layers. In particular, this first preliminary stretching is preferably greater than the preload with which the elastic strips, in the stretched state, are laminated with the nonwoven-fabric layers.

Due to the greater preliminary stretching, it is ensured that a particularly easy stretching of the elastic film or the strips formed therefrom is possible at least up to the stretching limit specified by the method. In this way, it is provided, for example, that the extruded elastic film or the strips cut therefrom are preliminarily stretched by 300% to 500% before being introduced between the nonwoven-fabric layers, wherein the length that results after a preload and that has a permanent deformation (permanent set) is to be used as the starting length for the subsequent stretching during the lamination process.

According to a refinement, to provide for a simple process, the two nonwoven-fabric layers are coated with hot melt adhesive, in particular, coated all over with hot melt adhesive, before the lamination process is carried out.

A disposable diaper can be formed using the above-described, elastic laminate for diaper closures. The disposable diaper comprises a basic diaper body which includes a fluid-absorbing core between a fluid-tight outer layer and a fluid-permeable inner layer, wherein two sections of the above-described elastic laminate are connected to the basis diaper body and are provided as a lateral connection between a front part and a rear part of the basic diaper body. In particular, each of the sections is connected at a first end to the basic diaper body and, at a second end, supports a closure element, for example, a hook material.

### BRIEF DESCRIPTION OF THE DRAWINGS

Further features and advantages of the invention will become apparent from the description of embodiments that follows, with reference to the attached figures, wherein:

FIG. 1 shows an elastic laminate for diaper closures, according to the invention, in a relaxed state;

FIG. 2 shows the elastic laminate shown to FIG. 1 in a fully stretched state at a stretching limit;

FIG. 3 shows a cross-section of the elastic laminate;

FIG. 4a shows a displacement-force diagram for a stretching of an elastic film provided for forming the elastic laminate;

FIG. 4b shows the area under the curve ascertained during the stretching in the displacement-force diagram in FIG. 4a;

FIG. 4c shows the area according to FIG. 4b for the return of the elastic film; and

FIG. 5 shows the schematic representation of a disposable diaper.

### DETAILED DESCRIPTION OF THE PREFERRED EMBODIMENTS

The following is a detailed description of example embodiments of the invention depicted in the accompanying drawings. The example embodiments are presented in such detail as to clearly communicate the invention and are designed to make such embodiments obvious to a person of ordinary skill in the art. However, the amount of detail offered is not intended to limit the anticipated variations of embodiments; on the contrary, the intention is to cover all modifications, equivalents, and alternatives falling within the spirit and scope of the present invention, as defined by the appended claims.

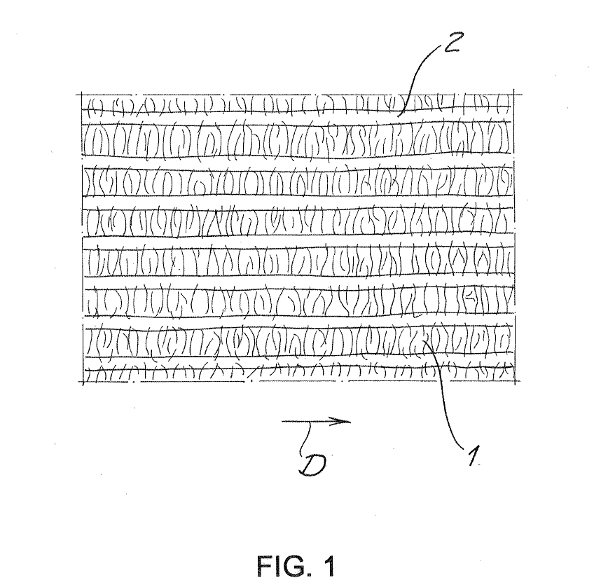

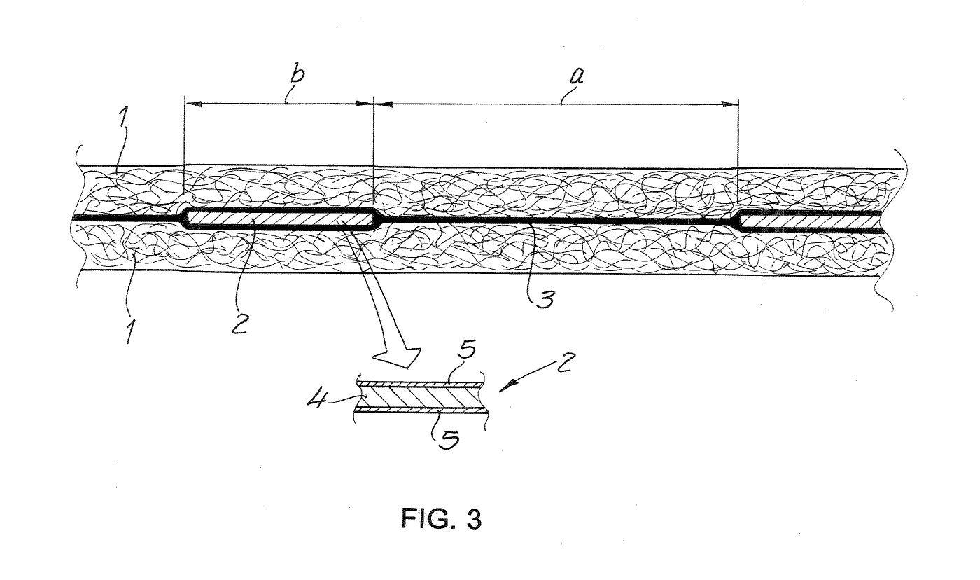

FIG. 1 shows a top view of an elastic laminate for diaper closures. When viewed in combination with the sectional representation according to FIG. 3, it is evident that the elastic laminate comprises two nonwoven-fabric layers 1, which also form the outer sides of the elastic laminate. Strips 2 of an elastic film, which are incorporated between the nonwoven-fabric layers 1 by an adhesive 3, extend along a stretching direction D.

According to the invention, the strips 2 are incorporated under preload between the nonwoven-fabric layers 1, wherein the nonwoven-fabric layers 1, in a relaxed state, represented in FIG. 1, are pleated along the stretching direction D due to the return force of the strips 2 which were incorporated under preload, i.e., said nonwoven-fabric layers are gathered along the strips 2. A t the point at which the strips 2 extend, the nonwoven-fabric layers 1 remain flat due to a preferably all-over adhesion by the adhesive, while ruffle-shaped gatherings form between the strips 2, which result in a highly non-uniform, soft surface of the elastic laminate. The elastic laminate therefore has a structure in the relaxed state that is very pleasant and is gentle on the skin.

The nonwoven-fabric layers 1, the strips 2, and the adhesive 3 are not shown to scale in FIG. 3. The thickness of the strips in the relaxed state is between 30 μm and 80 μm, wherein the width b determined perpendicularly to the stretching direction D is between 3 mm and 12 mm. The distance a measured from the edges of adjacent strips 2 also is between 3 mm and 12 mm, although the distance preferably is selected to be slightly greater than the width b in the specific exemplary embodiment, purely by way of example. In principle, the strips 2 of the elastic film in the relaxed state extend over 10% to 80% of the total area of the elastic laminate.

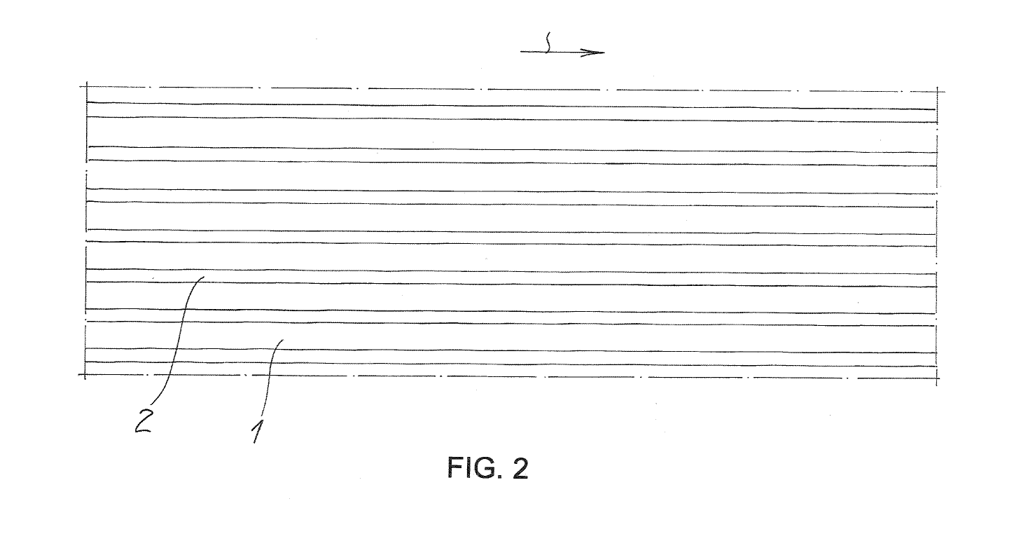

FIG. 2 shows the elastic laminate according to FIG. 1 in a stretched state. The extent of the stretching of the elastic laminate in this case corresponds to the tension of the strips of the elastic film during production. Correspondingly, the nonwoven-fabric layers 1 also are stretched again so far that the above-described pleating is eliminated again. The elastic laminate stretched along the course of the strips is therefore flat. The elastic laminate can be stretched further only by applying a substantially greater amount of force, because only those pleats that were previously formed via the contraction of the preloaded strips are pulled straight again, although without also changing the structure of the nonwoven-fabric layers, up to the point of the stretching and the flat arrangement of the nonwoven-fabric layers, which is shown.

A further stretching of the elastic laminate would result in a structural change and stretching of the nonwoven-fabric layers, i.e., a disproportionately greater amount of force must be applied for this purpose. From the perspective of a user, a stretching limit is clearly noticeable, wherein the overall stability of the elastic laminate at the stretching limit is substantially provided by the nonwoven-fabric layers.

Within the scope of the invention, this also enables the strips 2 of the elastic film to be designed to be particularly easily stretched and to not need to have a high tensile strength. With respect to the tensile strength and tear resistance on the one hand, and with respect to the elastic return on the other hand, a distribution of functions between the nonwoven-fabric layers 1 and the strips 2 of the elastic film is implemented.

According to FIG. 3, the film from which the strips 2 are formed can have a three-layered configuration comprising an elastic film layer 4 between two thin, non-elastic cover layers 5. The cover layers 5 are designed to be so thin and preferably also weakened by an activation that the elastic properties of the strips 2 and, therefore, of the elastic laminate formed therefrom, are determined by the elastic film layer 4. The elastic film layer 4 is formed based on styrene block copolymer. Specifically, the elastic film layer 4 consists of a first styrene-isoprene-styrene block copolymer and a second styrene-isoprene-styrene block copolymer, wherein the first styrene-isoprene-styrene block copolymer comprises a styrene portion between 10% and 20% by weight and has a hardness of less than 44 Shore A. The second styrene-isoprene-styrene block copolymer comprises a styrene portion between 30% and 40% by weight and has a hardness of more than 46 Shore A. The elastic film layer is formed without the addition of polystyrene and also contains less than 15%, preferably less than 10% additives, and particularly preferably contains approximately 6% additives.

Within the scope of the invention, the ratio of the first styrene-isoprene-styrene block copolymer to the second styrene-isoprene-styrene block copolymer is between 6:1 and 2:3; in the specific exemplary embodiment, this ratio is 3:1.

Due specifically to the first, very soft styrene-isoprene-styrene block copolymer and the omission of polystyrene in the formulation of the elastic film layer, a very soft stretching behavior also results in the machine direction, wherein a low tensile strength can be accepted, as described above.

It is assumed that the thin cover layers 5 also impart a stabilizing effect on the elastic film layer 4, as a core layer, during the co-extrusion.

The elastic film layer 4 has a thickness between 30 μm and 60 μm, for example 44 μm, while the cover layers formed from a mixture of polyethylene and polypropylene in the exemplary embodiment only have a thickness between 1.5 μm and 6 μm, for example 3μm.

In order to minimize a negative influence of the cover layers 5 on the elastic properties of the film and of the elastic laminate formed therefrom, the elastic film can be subjected to an activation. The activated film resulting upon discontinuation of the tensile forces is then used for forming the laminate, wherein the length that results after the activation and after the force is discontinued, and that may have a permanent deformation, is used as the starting length for the additional stretches.

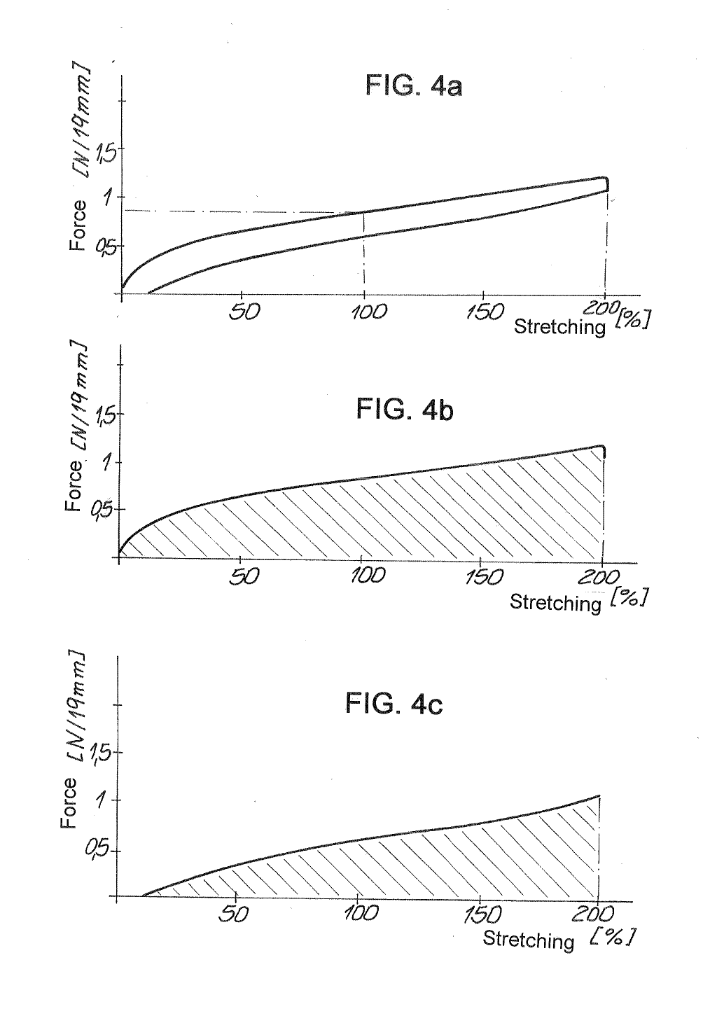

For example, FIG. 4a shows a displacement-force diagram for an elastic film that was initially activated by a stretching by 500%. FIG. 4a therefore shows the elastic behavior of the elastic film that has been activated in this way, starting from the new starting length which may have a permanent deformation. It is evident that the elastic film can be initially stretched by 100% using a low force of less than 1 N per 19 mm specimen width and can then be stretched by 200% using a force of less than 1.5 N per 19 mm specimen width. With the aid of the elastic film, an elastic laminate comprising cover layers made of a nonwoven fabric may therefore be produced, and the elastic laminate can be easily stretched along the strips 2 during the incorporation of strips of the elastic film in the stretched state up to the stretching limit established by this preliminary stretching.

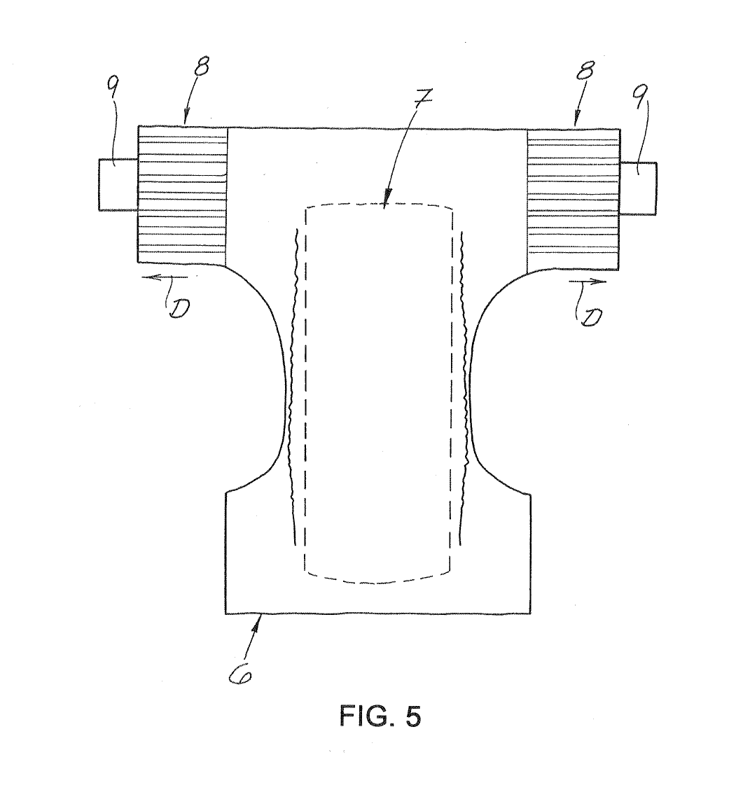

The strips 2 preferably are formed from the elastic film in such a way that the strips extend in the machine direction, i.e., the production direction, of the elastic film. FIG. 5 shows the preferred application of the elastic laminate according to FIGS. 1 and 2. FIG. 5 shows a disposable diaper comprising a basic diaper body 6. The disposable diaper 6 comprises a core 7 that absorbs a fluid between a fluid-tight outer layer and a fluid-permeable inner layer, wherein two sections 8 of the above-described elastic laminate are connected to the basic diaper body 6 and are provided as lateral connections between a front part and a rear part of the basic diaper body 6. Specifically, each of the sections 8 is connected at a first end to the basic diaper body 6 and, at a second end, support a closure element 9, for example, a hook material. The closure elements are intended to be fastened on a front section of the basic diaper body when the disposable diaper is worn, for which a contact surface, which is also referred to as a “landing zone”, is provided there for this purpose.

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

What is claimed is:

1. An elastic film, comprising:
an elastic film layer formed from a first styrene block copolymer and a second styrene block copolymer without the addition of polystyrene;
wherein the first styrene block copolymer has a styrene portion of less than 25% by weight and the second styrene block copolymer has a styrene portion of more than 26% by weight; and
wherein the ratio of the first styrene block copolymer to the second styrene block copolymer is between 6:1 and 2:3.

2. The elastic film according to claim 1, wherein the first styrene block copolymer has a hardness of less than 45 Shore A and the second styrene block copolymer has a hardness of more than 45 Shore A.

3. An elastic film, comprising:
an elastic film layer formed from a first styrene block copolymer and a second styrene block copolymer without the addition of polystyrene;
wherein the first styrene block copolymer has a styrene portion of less than 25% by weight and the second styrene block copolymer has a styrene portion of more than 26% by weight; and
wherein the ratio of the first styrene block copolymer to the second styrene block copolymer is between 5:1 and 1:1.

4. The elastic film according to claim 1, wherein the elastic film layer comprises a three-layered configuration, formed by co-extrusion, that is situated between two non-elastic cover layers.

5. The elastic film according to claim 4, wherein each of the non-elastic cover layers has a thickness between 1.5 μm and 6 μm.

6. The elastic film according to claim 4, wherein the non-elastic cover layers are formed from polyolefin;

7. The elastic film according to claim 6, wherein the polyolefin is polyethylene, polypropylene, or mixtures of polyethylene and polypropylene.

8. The elastic film according to claim 1, wherein the elastic film layer has a thickness between 30 μm and 60 μm.

9. The elastic film according to claim 1, defined by an energy recovery value of more than 0.6.

10. The elastic film according to claim 1, wherein after an initial activation by stretching by 500%, a force necessary for a stretching by 100% is less than 1 N per 19 mm specimen width.

11. The elastic film according to claim 1, wherein after an initial activation by stretching by 500%, a force necessary for a stretching by 200% is less than 1.5 N per 19 mm specimen width.

12. The elastic film according to claim 1, wherein a styrene-isoprene-styrene block copolymer is provided as the first and the second styrene block copolymers.

13. The elastic film according to claim 1, wherein the elastic film layer comprises, in addition to the first styrene block copolymer and the second styrene block copolymer, admixtures, additions and additives having an overall portion of less than 10% by weight.

14. A disposable diaper, comprising:
a diaper body comprising a front part, a rear part and a core configured to absorb a liquid, the core positioned between a fluid-tight outer layer and a fluid-permeable inner layer; and
first and second elastic laminate sections, each having first and second ends;
wherein the first ends of the first and second elastic laminate sections are connected to the diaper body and provided as lateral connections between the front part and the rear part of the diaper body; and
wherein first and second closure elements are connected to the respective second ends of the first and second elastic laminate sections.

15. The disposable diaper as set forth in claim 14, wherein the first and the second elastic laminate sections comprise an elastic film; and
wherein the elastic film comprises an elastic film layer formed from a first styrene block copolymer and a second styrene block copolymer without the addition of polystyrene;
wherein the first styrene block copolymer has a styrene portion of less than 25% by weight and the second styrene block copolymer has a styrene portion of more than 26% by weight; and
wherein the ratio of the first styrene block copolymer to the second styrene block copolymer is between 6:1 and 2:3.
